(19) United States
(12) Patent Application Publication
(10) Pub. No.: US 2004/0123350 A1
(43) Pub. Date: Jun. 24, 2004

(54) A METHOD FOR INCREASING AN ABIOTIC-RESISTANCE IN MONOCOT PLANT

(76) Inventors: Ju-Kon Kim, Seongnam (KR); Baek-Hie Nahm, Yongin (KR); Yang-Do Choi, Seoul (KR); In-Cheol Jang, Yongin (KR); Won-Bin Choi, Seoul (KR); Yeon-Seak Kim, Yongin (KR); Chung Ho Kim, Cheongju (KR); Sang-Ik Song, Sungnam (KR)

Correspondence Address: NATH & ASSOCIATES, 1030 15th STREET, 6TH FLOOR, WASHINGTON, DC, 20005, US

(21) Appl. No.: 10/324,058

(22) Filed: Dec. 20, 2002

(51) Int. Cl. A01H 1/00; C12N 15/82; A01H 5/00

(52) U.S. Cl. 800/289

(57) ABSTRACT

The present invention relates to a method for increasing resistance of monocot plants against abiotic stress which comprises a step of transforming monocot plants with a recombinant plasmid containing a fused gene (TPSP) of trehalose-6-phosphate synthetase (TPS) gene and trehalose-6-phosphate phosphatase (TPP) gene to express the TPSP gene while maintaining normal growth and development characteristics. The present invention can increase the resistance of monocot plants against various stresses so that it can greatly contribute to the improvement of production and quality of valuable agricultural crops.

### BACKGROUND OF THE INVENTION

1. Field of the Invention

The present invention relates to a method for increasing the resistance of monocot plants against abiotic stress. More specifically, the present invention relates to a method for increasing the resistance of monocot plants against abiotic stress by expressing trehalose-6-phosphate synthetase (TPS) and trehalose-6-phosphate phosphatase (TPP) in monocot plants while surprisingly maintaining normal growth and development characteristics.

2. Description of the Related Art

International Publication WO 00/70067, published Nov. 23, 2000, is directed to a rice actin 2 promoter and actin 2 intron and methods for the use thereof. Environment or stress resistance to drought (see corresponding U.S. Pat. No. 6,429,357 cols. 19 and 20) is described by introducing genes encoding for trehalose-6-phosphate synthase and through subsequent action of native phosphatases in the cell or by introduction and coexpression of a specific phosphatase resulting in trehalose which is a protective compound able to mitigate the effects of stress.

U.S. Pat. No. 5,925,804 is directed to the increase in the production of Trehalose in plants using an E. coli trehalose phosphate synthase gene, see cols. 7 and 8.

Seo H S et al., Appl. Environ. Microbiol., 66:2484-2490, (2000), which relates to the characterization of a bifunctional fusion enzyme (TPSP) of trehalose-6-phosphate synthetase and trehalose-6-phosphate phophatase of Escherichia coli.

Trehalose (α-D-glucopyranosyl-[1,1]-α-D-glucopyranose) is a non-reducing diglucoside and therefore does not react with amino acids or proteins as part of Maillard browning. Trehalose is found in various organisms, including bacteria, algae, fungi, yeast, insects and some plants, and serves not only as a carbohydrate reservoir but also as a protective agent against a variety of physical and chemical stresses (see, Elbein A, Adv. Carbohydr. Chem. Biochem., 30:227-256, 1974; Eleutherio E C A et al., Cryobiology, 30:591-596, 1993; Strom A R and Kaasen I, Mol. Microbiol., 8:205-210, 1993; van Laere A, FEMS Microbiol. Rev., 63:201-210, 1989; and Wiemken A, J. Gen. Microbiol., 58:209-217, 1990). Further, it has been known that trehalose shows a high water-retention activity under dry conditions to maintain the fluidity of the cell membranes and allow the plant to have a resistance against naturally occurring stresses during cycles of dehydration and rehydration (see, Leslie S B et al., Appl. Environ. Microbiol., 61:3592-3597, 1995; Drennan P M et al., J. Plant Physiol., 142:493-496, 1993; and Muller J et al., Plant Sci., 112:1-9, 1995). Such effect of trehalose on stress resistance has been demonstrated for cryptobiotic plant species such as S. leidophylla having resistance against dehydration. In this regard, it has been reported that trehalose accumulates to the level of 12% of plant dry weight during dehydration of such plant species, whereas trehalose accumulation is reduced during rehydration (see, Goddijn O J M and van Dun K, Trends Plant Sci., 4:315-319, 1999).

By virtue of such activity of trehalose, it has been attempted to increase stress resistance of plants. Up to the present, transgenic plants that express trehalose-6-phosphate synthetase (PTS) gene and/or trehalose-6-phosphate phosphatase (TPP) gene from E. coli or yeast in dicotyledon plants have been found. These transgenic plants express trehalose generally at a very low level. However, in these transgenic plants, although the stress resistance was somewhat increased, adverse effects appeared such as severe growth disturbance and warped roots. These adverse effects were exhibited even in the absence of trehalose accumulation (see, Holmstrom K-O et al., Nature, 379:683-684, 1996; Goddijn O J M et al., Plant Physiol., 113:181-1990, 1997; Muller et al., Plant Sci., 147:37-47, 1999; Pilon-Smits E A H et al., J. Plant Physiol., 152:525-532, 1998; and Romeo C et al., Planta, 201:293-297, 1997).

In the production of food for human welfare and existence, monocot plants, including rice, barley, wheat, maize, etc., are regarded as being commercially valuable plants. Therefore, a lot of effort has been exerted to increase the productivity and quality of such crops. Particularly, continuous efforts have been made in order to produce crops having resistance against abiotic natural conditions, such as drought, an increase in salt concentration, low temperature, etc.

Thus, the present inventors have earnestly studied to develop a method for increasing the resistance of monocot plants against abiotic stresses. As a result, the inventors have identified that when a fusion gene of trehalose-6-phosphate synthetase (TPS) gene and trehalose-6-phosphate phosphatase (TPP) gene is introduced and expressed in monocot plants, stress resistance of the plants against dehydration, high salt level and low temperature can be enhanced without inhibition of the growth level, and thus, completed the present invention.

Consequently, an object of the present invention is to provide a method for increasing resistance of monocot plants against abiotic stresses by expressing a fusion gene of TPS gene and TPP gene while maintaining phenotypic normalcy.

Another object of the present invention is to provide a method for producing monocot plants having increased resistance against abiotic stresses by expressing a fusion gene of TPS gene and TPP gene.

Another object of the present invention is to provide a method for producing monocot plants without morphological growth defects, such as growth and development disturbance and warped roots, and having increased resistance against abiotic stresses by expressing a fusion gene of the TPS gene and the TPP gene.

Another object of the present invention is to provide a method for producing monocot plants having increased resistance against abiotic stresses by expressing a fusion gene (TPSP) of the TPS gene and the TPP gene and which exhibit normal growth and development characteristics.

### SUMMARY OF THE INVENTION

The present invention relates to a method for increasing the resistance of monocot plants to better withstand abiotic stress, such as dehydration-stress, salt-stress or cold-stress, which comprises transforming a monocot plant with a recombinant plasmid containing a bifunctional fusion enzyme gene (TPSP) of the trehalose-6-phosphate synthetase (TPS) gene and the trehalose-6-phosphate phosphatase (TPP) gene to express the TPSP gene, thereby limiting the accumulation of trehalose-6-phosphate and enhancing the accumulation of trehalose in the transformed monocot plants to while maintaining normal growth characteristics.

Preferably, the TPS gene and TPP gene are derived from E. coli or yeast. The method according to the present invention can be used to increase the resistance of monocot plants, especially in the rice, wheat, barley and maize monocot plants, which are commercially important plants.

Introduction of the expressible bifunctional fusion gene into a recipient plant cell, i.e., transformation, is carried out according to Agrobacterium-mediated method.

### BRIEF DESCRIPTION OF THE DRAWINGS

FIG. 1 is a drawing showing the map of TPSP gene as the fused recombinant gene of TPS and TPP;

FIG. 2 is a drawing showing the gene map of recombinant plasmid pSB-UTPSP;

FIG. 3a is the standard HPIC chromatogram of trehalose;

FIG. 3b is HPIC chromatogram showing a carbohydrate profile of Ubi1::TPSP plant leaves;

FIG. 3c is HPIC chromatogram showing a carbohydrate profile of the extract of Ubi1::TPSP plant seeds;

FIG. 4a is a graph showing chlorophyll fluorescence in dehydration-stress treated Ubi1::TPSP rice plants;

FIG. 4b is a graph showing chlorophyll fluorescence in salt-stress treated Ubi1::TPSP rice plants; and

FIG. 4c is a graph showing chlorophyll fluorescence in cold-stress treated Ubi1::TPSP rice plants.

### DETAILED DESCRIPTION OF THE INVENTION

According to the present invention, tie method for increasing resistance of monocot plants against abiotic stresses comprises a step of transforming monocot plants with a recombinant plasmid containing a fused gene (TPSP) of trehalose-6-phosphate synthetase (TPS) gene and trehalose-6-phosphate phosphatase (TPP) gene to express TPSP gene. In this method, the TPS gene and TPP gene are derived from E. coli or yeast and introduced into monocot plants such as rice, barley, wheat or maize by means of the Agrobacterium-mediated method. Although the abiotic stresses in this method are not particularly restricted, they can include dehydration-stress, salt-stress or cold-stress.

The method for producing monocot plants having increased resistance against abiotic stresses according to the present invention is conducted through the same steps as the method for increasing stress of monocot plants against said abiotic stresses, except that the recombinant plasmid containing TPSP gene is introduced into monocot plants or their ancestor cells as long as the cells are capable of being regenerated into plants.

Hereinafter, the present invention will be more specifically explained.

Up to the present, attempts to increase stress resistance in plants using trehalose has been done in only dicotyledon plants due to difficulties including the absence of suitable vectors for monocot plants and a deficiency of research papers. However, although the expression of trehalose increases the stress resistance in dicotyledon plants, it could not obtain any remarkable effect due to the side effect of severely inhibiting the growth of plants. Thus, such attempt has never been made in monocot plants as well.

In order to increase stress resistance in monocot plants as valuable food resources, the present inventors incorporated a fusion gene of genes coding for trehalose-6-phosphate synthetase (TPS) and trehalose-6-phosphate phosphatase (TPP), both of which are the enzymes required for trehalose synthesis, into a vector containing Ubi1 promoter exhibiting a relatively high activity in monocot plants to construct the recombinant plasmid, which was then introduced into rice plants by means of transformation mediated by Agrobacterium tumefaciens.

Then, the transformed rice genotypes were analyzed with Southern Blot to identify that introduced genes were stably integrated into the rice chromosomes. Further, rice RNAs. extracted from said rice leaves were analyzed with Northern Blot to identify that the genes introduced were normally expressed. In addition, it has been confirmed through carbohydrate quantitative analysis that trehalose was expressed at as high a level as 200 times the expression level known from tobacco transformed with TPS or TPP in the prior art. The observation at the level of cultivation revealed that contrary to dicotyledon plants, the overexpression of trehalose in rice plants does not greatly affect the growth of rice as monocot plants, and further, it has also been identified that trehalose results in increasing the resistance against abiotic stresses, such as dehydration, salt and low temperature.

Accordingly, it is expected that the method of the present invention can largely contribute to the production and quality improvement of valuable agricultural crops since it can increase the resistance of monocot plants against various stresses. Hereinafter, the present invention will be more specifically illustrated through the following examples. A person having an ordinary knowledge in the relevant technical field will understand that these examples are intended only to specifically explain the present invention and the scope of the present invention is not limited by these examples.

### EXAMPLE 1

### Construction of Plasmid and Transformation of Rice Plants

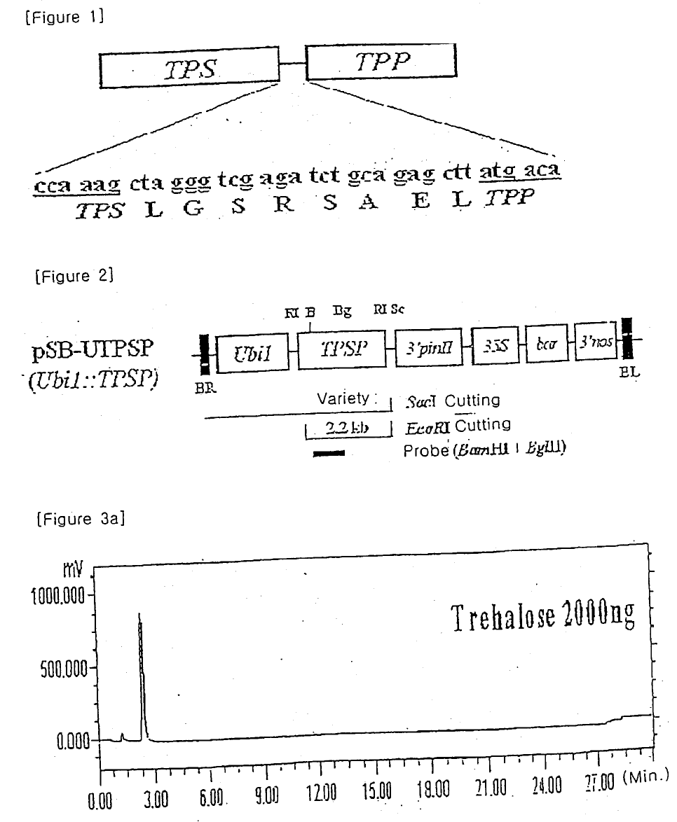

The stop codon of E. coli TPS gene was removed through PCR and then ligated with TPP gene to construct TPSP as the fusion recombinant gene of TPS and TPP. (See, FIG. 1 and Seo H S et al., Appl. Environ. Microbiol., 66:2484-2490, (2000), which is incorporated by reference as if fully described herein.) The resulting TPSP was linked to maize ubiquitin promoter to construct Ubi1::TPSP, which was inserted into the expression vector containing 35S promoter and bar coding region (phosphinothricin acetyltransferase gene) to construct recombinant plasmid pSB-UTPSP (see, FIG. 2). FIG. 2 is the diagram showing the gene map of recombinant plasmid pSB-UTPSP, wherein BR represents a right-border sequence; BL represents a left-border sequence; 3′ pinII represents the 3′-region of potato protease inhibitor II gene; 35S represents 35S promoter; and 3′ nos represents the 3′-region of nopaline synthase gene. Since phosphinothricin acetyl transferase encoded into bar gene functions to detoxify the toxicity of phosphinothricin-derived herbicides, it can act as a selective marker. The pSB-UTPSP was introduced into Agrobacterium tumefaciens LBA4404 by triparental mating.

For transformation of rice plants with said Agrobacterium tumefaciens LBA4404, 70% (v/v) ethanol was added to about 200 unhulled seeds (Oryza sativa L. cv Nakdong) and gently mixed together for one minute to sterilize the seeds. Then, ethanol was discarded and the seeds were further sterilized by gentle mixing with 100 ml of 20% (v/v) Clorax for one hour, and then washed several times with sterilized water. Callus induction from the seeds, co-cultivation of callus with Agrobacterium containing the plasmid constructed as described above, and the selection of transformed callus were carried out as previously described (see, Jang, I-C. et al., Mol. Breeding, 5:453-461, 1999). Rice plants transformed with Agrobacterium-mediated method were cultivated in a greenhouse to select only the plants having resistance against the herbicide Basta. According to Southern blot analysis of transgenic rice plants transformed and selected as described above, it could be identified that the introduced transgene was integrated into rice chromosomes and had one to three copy numbers. For further tests, the plants containing a single copy of TPSP gene were selected, and Northern blot analysis using total RNA samples from leaves of the selected plants could observe mRNA of approximately 2.4 kb, thereby it was identified that TPSP was normally expressed.

### EXAMPLE 2

### Investigation of Accumulation Level of Trehalose in Transgenic Plants

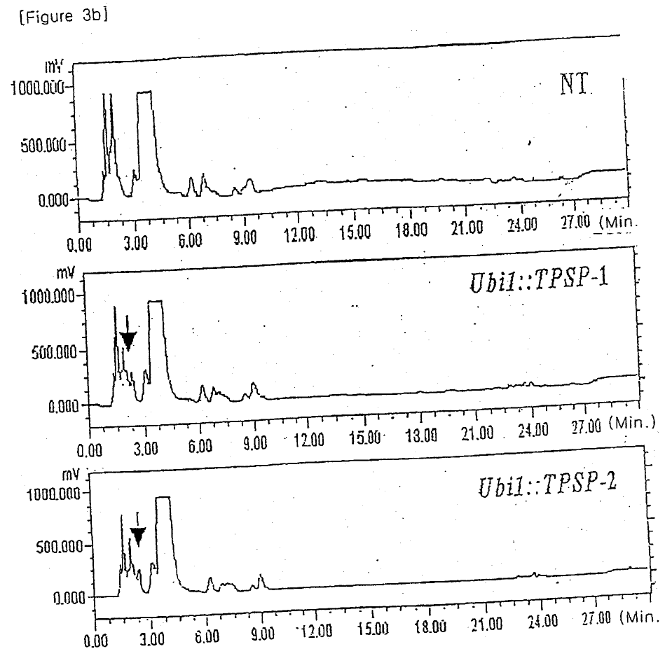

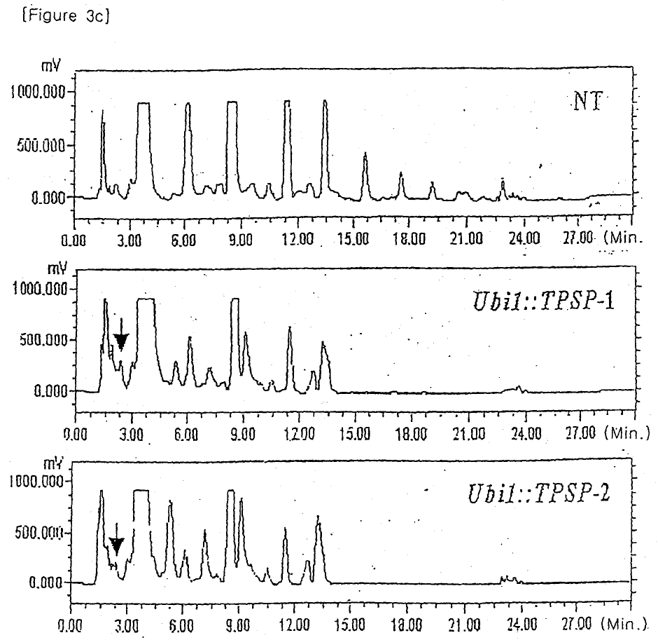

To investigate the accumulation level of trehalose in transgenic plants and the effect of trehalose on the carbohydrate content in plants, leaves and seeds of Ubi1::TPSP as the transgenic rice plants produced in Example 1 were digested in liquid nitrogen and then extracted with 10 ml/g of water at 100° C. for 10 minutes. The extract was centrifuged, and the resulting supernatant was filtered through a 0.45 Ñ m filter. Then, the quantitative analysis of carbohydrate was carried out by means of DX500 HPIC (high performance ion chromatography, Dionex 500, Dionex, USA) equipped with a 4×250 nm Carbo-Pak PA1 column. HPIC was carried out under a linear gradient condition using 150 mM NaOH solution containing 0 mM to 250 mM sodium acetate for 30 minutes. The HPIC result was monitored with ED40 electrochemical detector (Dionex DC Amperometry, Dionex, USA) using commercially available trehalose (Sigma Chemicals Co., USA) as the standard (see, FIG. 3a).

The effects of trehalose on the composition and distribution of respective carbohydrates are shown in FIGS. 3b and 3c. FIGS. 3b and 3c are HPIC chromatograms showing carbohydrate profiles in the extracts of Ubi1::TPSP plant leaves and seeds, respectively, wherein NT represents untransformed rice plant, Ubi1::TPSP-1 and Ubi1::TPSP-2 represent two plants containing a single copy number of TPSP gene as selected in Example 1. As can be seen from FIGS. 3b and 3c, trehalose was present in the leaf and seed extracts of Ubi1::TPSP rice plants at the level of about 1.076 mg/g, which is 200-fold higher than the level known from transgenic tobacco plants transformed with TPS or TPP genes. Further, it could also be identified that the carbohydrate content was substantially not altered in the leaf extract of Ubi1::TPSP rice plants but was greatly altered in the seed extract.

In addition, as the result of observation for the cultivation level of Ubi1::TPSP rice plants, it could be identified that Ubi1::TPSP rice plants grew up to a level similar to untransformed rice plants. Up to the present, transgenic plants transformed with TPS and/or TPP of E. coli or yeasts have been known for dicotyledon plants, and it has been reported in these transgenic plants that although trehalose is expressed at a very low level, there occurred such phenomena as severe disturbance of growth and development and warped roots. However, Ubi1::TPSP rice plants did not show any change in root appearance as well as in their growth even though they excessively produced trehalose at the level of 0.1% of the plant mass. Accordingly, it could be found that contrary to dicotyledon plants the overexpression of trehalose in rice plants does not inhibit the normal growth of plants.

### EXAMPLE 3

### Increase of Stress-Resistance by Trehalose

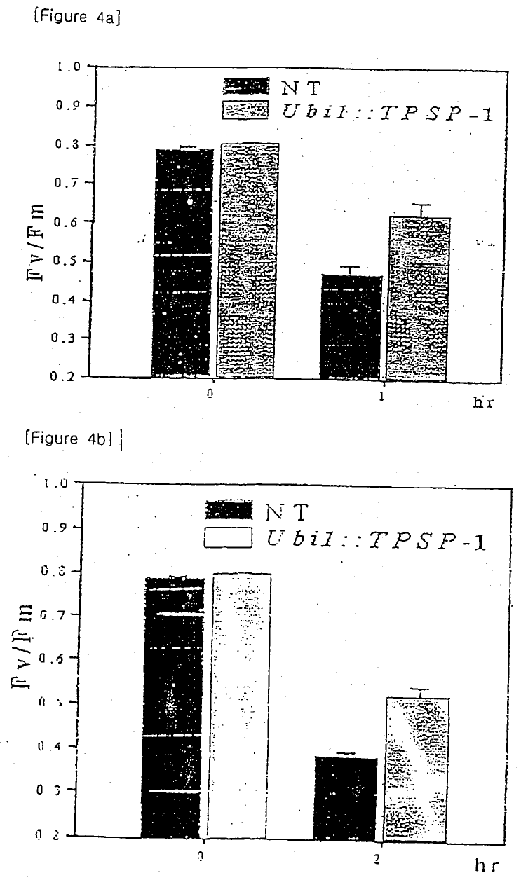

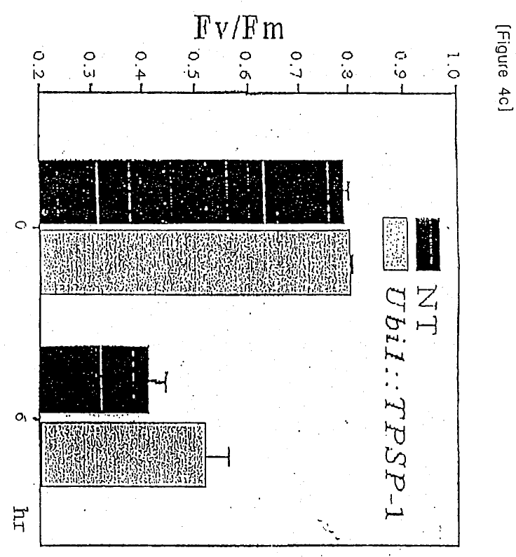

The seeds, sterilized with ethanol and Clorax and washed as described in Example 1, were germinated on soil in a growth chamber at 28° C. with cycles of 16 hours light/8 hours dark conditions and then grown for 14 days to produce the young seedlings. For the dehydration-stress treatment, whole plants were air-dried for one hour at 28° C. under light condition of 150 Ñ m/m2/s. For the salt-stress treatment, said young seedlings were grown in a nutrient solution of 0.1% (v/v) Hyponex (Hyponex, Japan) for 2 days, transferred to a fresh nutrient solution containing 9% (w/v) NaCl and then grown under light condition of 150 Ñ m/m2/s for 2 hours at 28° C. For the cold-stress treatment, said young seedlings were grown under light conditions of 150 Ñ m/m/2s for 6 hours at 4° C. Then, the untransformed control group and the transgenic test groups with stress treatment under various conditions were kept for 2 hours under dark condition and their chlorophyll fluorescent levels were measured using a pulse modulation (PAM) fluorometer. The chlorophyll fluorescent level was represented by the ratio (Fv/Fm) of measured minimum fluorescence (Fv) to maximum fluorescence (Fm), wherein the Fv/Fm ratio means the activity of photosystem II, and therefore, can be used as a measure to assess the functional damage of the plants (see, FIGS. 4a, 4b and 4c).

FIGS. 4a, 4b and 4c are the graphs showing chlorophyll fluorescence in dehydration-, salt- and cold-stress treated Ubi1::TPSP rice plants. As can be seen from FIGS. 4a, 4b and 4c, all rice plants treated with dehydration-, salt- and cold-stress showed the Fv/Fm ratios at the level, which is 15-19% higher than that in the control group. Accordingly, it could be confirmed that trehalose plays a role to increase the resistance of rice plants against abiotic stresses.

Effect of Invention

As explained and demonstrated above, the present invention relates to a method for increasing resistance of monocot plants against abiotic stress which comprises a step of transforming monocot plants with a recombinant plasmid containing a fused gene (TPSP) of trehalose-6-phosphate synthetase (TPS) gene and trehalose-6-phosphate phosphatase (TPP) gene to express TPSP gene. The present invention increases the resistance of monocot plants against various stresses so that it can greatly contribute to the improvement of production and quality of valuable agricultural crops.

What is claimed is:

1. A method for increasing resistance of monocot plants against abiotic stress, which comprises a step of transforming a monocot plant with a recombinant plasmid containing a fused bifunctional fusion enzyme gene (TPSP) of trehalose-6-phosphate synthetase (TPS) gene and trehalose-6-phosphate phosphatase (TPP) gene to express the TPSP gene, thereby limiting trehalose-6-phosphate accumulation and enhancing accumulation of trehalose in the transformed monocot plants while maintaining normal plant growth and development characteristics.

2. The method for increasing resistance of monocot plants against abiotic stresses according to claim 1, wherein TPS gene and TPP gene are derived from E. coli or yeast.

3. The method for increasing resistance of monocot plants against abiotic stresses according to claim 1, wherein the monocot plant is rice, wheat, barley, wheat or maize.

4. The method for increasing resistance of monocot plants against abiotic stresses according to claim 1, wherein the transformation is carried out according to Agrobacterium-mediated method.

5. The method for increasing resistance of monocot plants against abiotic stresses according to claim 1, wherein the abiotic stress is dehydration-stress, salt-stress or cold-stress.

6. A method for producing monocot plants having increased resistance against abiotic stresses, which comprises a step of transforming monocot plants or their ancestors with a recombinant plasmid containing a fused bifunctional fusion enzyme gene (TPSP) of trehalose-6-phosphate synthetase (TPS) gene and trehalose-6-phosphate phosphatase (TPP) gene to express the TPSP gene, thereby limiting trehalose-6-phosphate accumulation and enhancing accumulation of trehalose in the transformed monocot plants to enable growth without phenotypic growth alteration.

7. A method for generating a transgenic monocot plant comprising:
(a) constructing a (TPSP) fusion gene sequence consisting of a fused trehalose-6-phosphate synthetase (TPS) gene and trehalose-6-phosphate phosphatase (TPP) gene;
(b) transforming said TSPA fused gene sequence into a recipient plant cell; and
(c) regenerating said plant cell into a mature plant, wherein said mature plant is a transgenic monocot plant comprising said (TPSP) fusion gene sequence.

8. The method of claim 7 wherein the transgenic monocot plant is selected from the group of monocot plants consisting of; rice, wheat, barley or maize.

9. A transgenic monocot plant produced by the method of claim 7.
